(19) United States
(12) Patent Application Publication
(10) Pub. No.: US 2022/0054321 A1
(43) Pub. Date: Feb. 24, 2022

(54) ELASTIC SHEET MATERIAL, BANDAGE MADE THEREOF AND METHOD FOR PRODUCING SAID MATERIAL

(71) Applicant: Ottobock SE & Co. KGAA, Duderstadt (DE)

(72) Inventors: Helle HORMES, Northeim (DE); Olaf Kroll-Orywahl, Northeim (DE); Marcel Jung, Duderstadt (DE); Mario Koppe, Göttingen (DE); Hans-Peter Müller, Oberkotzau (DE); Christoph Schulz, Gerblingerrode (DE); Christopher Lee Lübbers, Göttingen (DE)

(73) Assignee: Ottobock SE & Co. KGAA, Duderstadt (DE)

(21) Appl. No.: 17/414,942

(22) PCT Filed: Dec. 4, 2019
(86) PCT No.: PCT/EP2019/083657

§ 371 Date: Jun. 16, 2021

(30) Foreign Application Priority Data
Dec. 18, 2018 (DE) 10 2018 132 640.8

(51) Int. Cl. A61F 13/08; A61F 2/78; A61F 13/06; A61F 13/00

(52) U.S. Cl. CPC A61F 13/08; A61F 2/78; A61F 13/061; A61F 2002/7837; A61F 13/00029; A61F 13/00017; A61F 13/00038

(57) ABSTRACT

The invention relates to an elastic sheet material having a front side, a rear side opposite the front side and at least one functional element, which is arranged on the front side and is connected to the sheet material. A limiting element for limiting the elasticity of the sheet material is arranged on the rear side of the sheet material opposite the functional element and/or on the front side underneath the functional element or within the sheet material underneath the functional element.

The invention relates to an elastic sheet material with a front side, a rear side situated opposite the front side, and at least one functional element arranged on the front side and connected to the sheet material. The invention likewise relates to a bandage made of such a sheet material and to a method for producing such a sheet material. Such a sheet material is used in particular as starting material for producing orthopedic equipment or orthoses, in particular for producing sleeves or bandages.

Sleeves, bandages or orthoses generally have an elastic basic body which can be placed around the limb or a joint area. A stabilizing element, for example a metal rail or a plastic rail, may be arranged on the basic body. Bandages or sleeves are applied to support or stabilize the joint function or the muscles and ligaments. For this purpose, the bandage is pulled over or wound around the joint to be protected or the muscles to be supported and is secured. In contrast to immobilizing orthoses, bandages allow the limbs connected by the joint to move relative to one another. For this purpose, the bandages have a basic body consisting of a flexible material, for example a foam or a textile. To support the stabilization action, stabilizing elements which have greater strength and stiffness than the basic body are arranged on the basic body, with the result that, in addition to protection against mechanical influence, an increased restriction on movement is obtained.

US 2008/0039757 A1 discloses a flexible bandage with a framework having a surface for placing on an area of a body, wherein the framework has a multiplicity of openings. A woven fabric or a foam may be attached to the framework itself, for example by welding, adhesive bonding or sewing.

EP 876 130 B1 describes an orthopedic brace with a retainer made of a unipartite injection-molded part having regions of different thicknesses, in order to adapt to the contour of a selected part of a limb. A layer of elastic material is attached inside the retainer, in order to cushion the retainer.

U.S. Pat. No. 6,024,712 A describes an ankle-joint orthosis with an inner textile part extending at least partially around an ankle joint. An outer brace is connected to the textile by an injection-molding process and constitutes additional support for the joint against unintended movements.

CA 2,398,059 A1 describes an orthopedic brace with a flexible inner part and an outer brace which is molded directly onto the flexible inner part.

WO 2013/072064 A1 describes an orthopedic sleeve for placing on a body, which orthopedic sleeve has a flexible basic body with at least one stabilizing element made of plastic arranged thereon. The stabilizing element has regions with a reduced cross section in comparison with the rest of the cross section, in which regions the stabilizing element is adhesively bonded on the basic body and/or materially bonded to the basic body. The stabilizing element may be applied to a flexible carrier material, in particular molded-on or back-molded on said flexible carrier material. In that case, the carrier material forms the regions which are materially bonded to the basic body.

When connecting polymers to elastic, in particular textile sheet materials, frequently used is an injection-molding process, in which the polymers penetrate deeply into the textile woven fabric. A problem in the case of this production process is the lack of flexibility, since a new mold has to be laboriously produced for each design.

It is therefore an object of the present invention to provide a sheet material, a bandage, and a method for producing a sheet material which allow a permanent and cost-effective connection of a functional element to the sheet material with a high rate of individualization.

According to the invention, said object is achieved by a sheet material having the features of the main claim, a bandage produced therefrom, and a method having the features of the additional independent claim. Advantageous embodiments and refinements of the invention are disclosed in the respective dependent claims, the description and the figures.

The elastic sheet material according to the invention with a front side, a rear side situated opposite the front side, and at least one functional element arranged on the front side and connected to the sheet material provides that a limiting element for limiting the elasticity of the sheet material, in particular locally in the region of the functional element, is arranged on the rear side of the sheet material opposite the functional element and/or on the front side below the functional element and/or inside the sheet material. The rear side of an elastic sheet material is used in particular to abut or bear against a skin surface, while a functional element generally has a rigid or protruding structure which is better arranged on the outer side, that is to say on that side of the sheet material which faces away from the body. The arrangement of a limiting element, which limits or nullifies the elasticity of the sheet material, at least in the plane of the front side and rear side has the advantage that, even when the material of the functional element penetrates less deeply into the structure of the elastic sheet material, there is no tensile stress and no relative movement in the case of stretching and contracting of the textile in the case of radial or longitudinal widening of the sheet material. A reduction in the thickness of the sheet material, that is to say a movement of the rear side toward the front side, is harmless in this respect and does not need to be prevented by the limiting element. A limitation of the elasticity predominantly in one direction can be achieved by specific embodiments of the limiting element, for example by a flexible woven fabric which is rigid under tension in one direction and elastic in the direction oriented perpendicular thereto. In this way, a sufficient limitation is achieved, in particular in the case of functional elements extending predominantly only in one direction. The limiting element prevents or lessens stretching and contracting of the sheet material as carrier for the functional element and thus prevents or reduces a relative movement between the functional element and the sheet material, since no tensile stresses or lessened tensile stresses are transferred to the binding surfaces or the binding region of the functional element to the sheet material when the sheet material is stretched. This prevents a situation in which the physical connection between the functional element and the sheet material becomes loose or is nullified, with the result that the fatigue strength of the bond of the functional element to the sheet material is maintained. Surprisingly, it has been found that, even when there is a stiffening only on one side of the sheet material, that is to say on the front side, inside the sheet material or on the rear side, the fatigue strength of the connection of the functional element to the sheet material has substantially increased, since the sheet material has been partially stiffened beforehand by virtue of the application or arrangement of the limiting element. Moreover, the partial rigidity of the sheet material has the advantage that the elasticity of the end product in its entirety is restricted only negligibly and only in the partial regions in which the functional element is arranged. Since the functional elements are generally likewise of rigid configuration in the region of the bond to the elastic sheet material, no notable restriction in terms of the wearing comfort of the sheet material is produced when said sheet material is processed to form an orthopedic device, for example a bandage or a sleeve. The limiting element may be arranged on the side situated opposite the functional element as well as form an intermediate layer between functional element and sheet material.

The sheet material is preferably in the form of a textile, for example a knitted fabric, laid fabric or woven fabric. An embodiment as a spacer knit is likewise provided. As an alternative, the sheet material may be in the form of foam or contain foam fractions. A sheet material is considered to be a material which has a predominantly two-dimensional extent, that is to say a large surface with a low material thickness.

In one refinement of the invention, the limiting element has an outer contour which corresponds to the outer contour of the functional element or at least partially, preferably completely, encloses the outer contour of the functional element. If the outer contour of the limiting element corresponds to the outer contour of the functional element, only a minimum region of the sheet material is stiffened, as a result of which the remaining elastic behavior of the sheet material is not impaired. With a surface region of the limiting element that is enlarged in comparison with the outer contour of the functional element, a larger surface of the sheet material is stiffened, this on the one hand facilitating the positioning of the functional element and on the other hand preventing tensile stresses in the peripheral region of the sheet material at the transition to the functional element and thus providing increased fatigue strength. As an alternative, the outer contour of the limiting element may also cover only those regions of the functional element which have a rigid form. Other regions of the functional element, for instance springs, which themselves generate sufficient elasticity may follow the movement of the elastic sheet material and do not come off quickly enough for a limitation of the elasticity of the sheet material to be imperatively necessary here.

The limiting element is preferably materially bonded, for example welded or adhesively bonded, to the sheet material.

The limiting element may be adhesively bonded or welded, preferably adhesively bonded or welded over its entire surface area, to the sheet material in the form of a film. It is likewise possible for the limiting element to be formed by an adhesive layer or by a multiplicity of adhesive strips or dots of adhesive or a grid of adhesive, which is applied to the sheet material such that it adheres to the surface and/or penetrates into the sheet material. As an alternative, the limiting element may be in the form of an inelastic but flexible textile, a penetrating element which penetrates the sheet material and forms a projection on the opposite side, in particular the front side, a conductive textile or band or cable, for example having a metallic thread component, certainly in order to allow sufficient flexibility, but also to at least reduce or nullify the elasticity in the partial region in which the limiting element is applied. The limiting element may also be in the form of a sensor, for example a capacitive sensor, in order to provide a functional contribution to the configuration of an orthopedic component beyond the limiting function in terms of elasticity.

One refinement of the invention provides that a plurality of limiting elements are arranged separate from one another on the sheet material, in order to not restrict or to restrict as little as possible the elasticity and flexibility of the entire sheet material. The limiting elements may be applied and fixed to the sheet material separate from one another and one behind another in a longitudinal extent, for example. In the case of a sheath-like embodiment of the finished product, it is likewise possible to provide limiting elements which are oriented in the longitudinal direction distributed over the circumference and allow a radial widening in the intermediate regions between the limiting elements but prevent an elongation in the longitudinal extent at least in certain regions.

The limiting elements or the limiting element preferably have a flexible form, in order to be able to adapt the sheet material to different surfaces, in particular body surfaces. This means that the limiting element is pliable, but does not permit a change in length or only permits a lessened change in length in the plane.

One refinement of the invention provides that the functional element is printed on the sheet textile or applied to the sheet material by means of an additive production process, that is to say is applied to the sheet textile in a layer-by-layer deposition process or additive production process and form-fittingly connected and/or materially bonded there. The functional element may be in the form of an elastomer or may be formed from an elastomer, for example, and in particular applied to the textile in an additive production process. Additive production processes, e.g. what is known as 3D printing, for example by means of an SLA printer or FDM printer, make it possible to produce individual products by applying, e.g. printing, the functional elements onto the sheet material in a manner adapted to the respective user and then process it to form an end product. It is also possible to bring the sheet material firstly into a basic form, for example in the form of a knee sleeve, elbow sleeve or calf sleeve, and then to apply or print the respectively desired functional element onto the already preformed basic form. The application or printing may be performed after adapting the bandage to the respective body, for example, with the result that the positioning of the functional elements on the sheet textile can be performed to high precision and individually.

The functional element may be in the form of a spring, rail, stiffening element, component receptacle, duct, hook, sensor mount, cable guide, cable, conductor, clip element, clasp, zip fastener, protector, cushion, air cushion, elastomer element and/or sensor.

The invention likewise relates to a bandage made of a sheet material as has been described above. One refinement of the bandage provides that it has a sheath-like form with a closed cross section or is formed from a sheet-like basic body with connecting devices arranged at opposite end regions. If the bandage has a closed cross section, it may have an interrupted form, for example in order to form recesses for projections. In the case of an embodiment of the bandage as a knee bandage, a recess may be formed in the region of the patella and/or hollow of the knee; in the case of an elbow bandage, a recess may be arranged for example in the region of the bend of the elbow. With a closed cross section, the bandage can be pulled over a limb. As an alternative, it is possible to form the bandage with an open cross section, to close it after being placed onto the body and secure it in the respective desired position and with the desired tension. For this purpose, connecting devices such as hook and loop fasteners, loops or clasps are arranged at opposite edges or end regions of the sheet material, in order to close the initially open cross section of the blank.

The method for producing a sheet material described above provides that the functional element is connected to the sheet material and at least one limiting element is arranged and connected between the functional element and the sheet material and/or inside the sheet element, in particular in the region of the functional element, and/or on that side of the sheet material which is situated opposite the functional element, and the limiting element at least reduces the elasticity of the sheet material. In the process, the limiting element is connected to the sheet material preferably before the functional element is connected to the sheet material.

The functional element may be applied or printed onto the sheet material or the limiting element in an additive production process, e.g. 3D printing process. It is preferable if the limiting element is connected to the sheet material over its entire surface area, for example welded or adhesively bonded over its entire surface area, in order to prevent a loosening and relative movement of the sheet material that occurs in the binding region of the functional element.

The functional element is preferably arranged inside the outer contour of the limiting element, to allow no relative movements between the sheet material and the functional element at the transition points of the functional element to the sheet element and thus to increase the fatigue strength of the fastened functional element.

The limiting element is preferably materially bonded to the sheet material, in order to achieve a permanent limitation of the elasticity.

The invention will be explained in more detail below on the basis of the figures, in which:

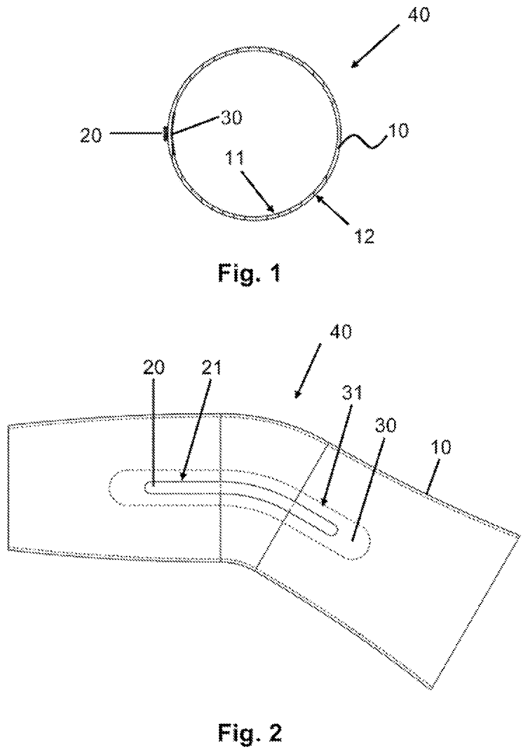

FIG. 1 shows a sectional illustration of a bandage;

FIG. 2 shows a side view of a knee bandage;

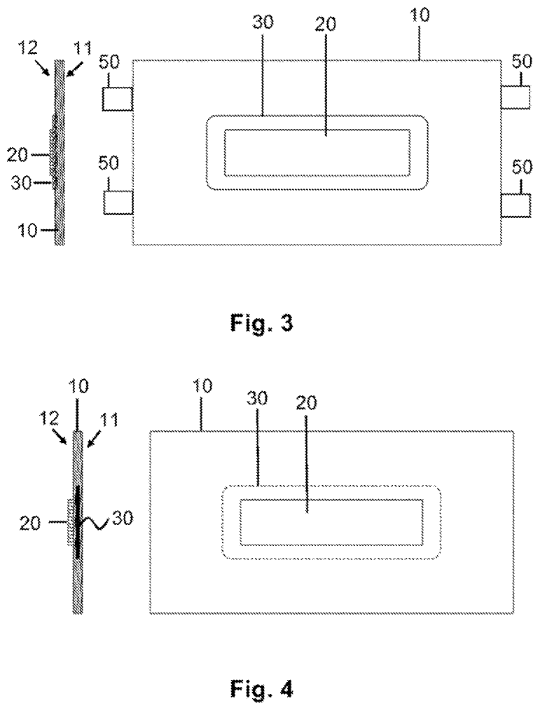

FIG. 3 shows a sectional illustration and a plan view of a sheet material;

FIG. 4 shows a variant of FIG. 3;

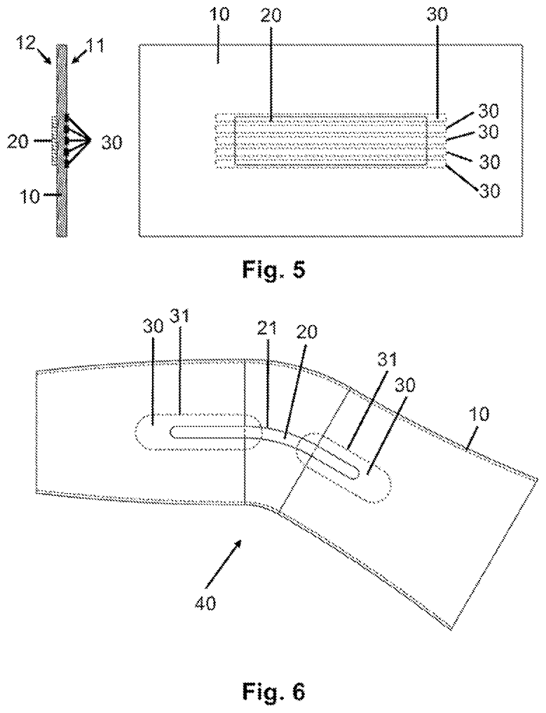

FIG. 5 shows a variant of FIG. 3;

FIG. 6 shows a variant of FIG. 2;

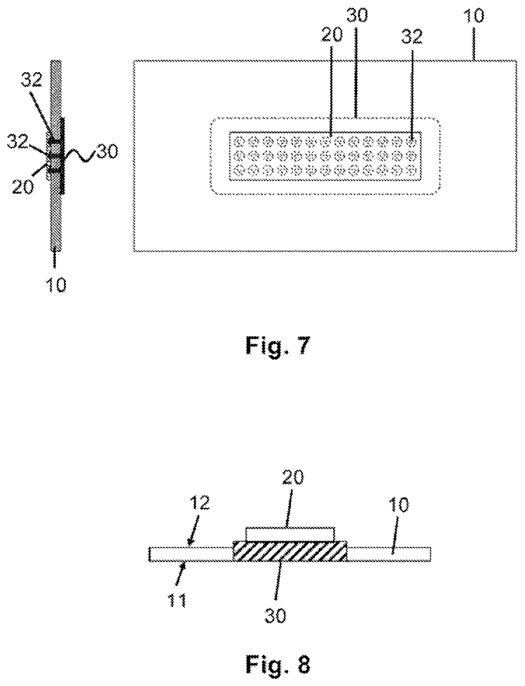

FIG. 7 shows a variant of FIGS. 5; and

FIG. 8 shows a sectional illustration through a sheet material.

FIG. 1 shows a cross section through a sheath-like bandage 40, which has a basic body made of a sheet material 10. The sheet material 10 is produced from an elastic material, in particular an elastic textile or a foam, and in the exemplary embodiment illustrated has a closed cross section, with the result that the bandage 40 overall has a sheath-like form. The cross section and the form of the basic body may vary in the course of the longitudinal extent of the bandage 40. The elastic sheet material 10 has an inner side which faces toward the user or patient and is referred to as the rear side 11, and an outer side or front side 12 which faces away from the patient in the applied state of the bandage 40 and is situated opposite the rear side 11. Arranged on the front side 12 of the basic body is a functional element 20, which is connected to the sheet material 10 of the basic body. Arranged on the rear side 11 opposite the functional element 20 is a limiting element 30, which serves to limit the elasticity of the sheet material 10. The limiting element 30 locally reduces, if appropriate reduces to zero, the stretchability of the sheet material 10 in the region of the functional element 20, with the result that, when the elastic sheet material 10 is being loaded owing to a movement or owing to the placement of the bandage 40 onto the patient, no or virtually no stretching occurs in the region of the functional element 20; however, the stretchability in the rest of the regions of the bandage 40 is not impaired. As a result, the application of shear stresses in the region of the connection between the functional element 20 and the sheet material 10 is prevented or at least significantly lessened, and therefore the functional element 20 can be fastened securely and permanently to the outer side or front side 12 of the sheet material 10.

FIG. 2 shows one embodiment and use of the elastic sheet material 10 as a bandage 40, which is intended to span a knee joint. The bandage 40 is configured in the form of a sheath and has a preflexed portion in order to prevent excessive stretching in the kneecap area, that is to say on the front side of the knee, and excessive compression in the area of the hollow of the knee, that is to say on the rear side of the knee. The lower-leg area, shown on the left in FIG. 2, has a cross section which narrows toward the distal end; the upper-leg area widens toward the proximal end. A functional element 20 configured as a spring, rail or a stiffening element is applied, in particular welded or adhesively bonded, to the outer side so as to span the knee joint. The functional element 20 may also be in the form of a duct or component receptacle for receiving a rail, a spring or an articulated rail; it may likewise be in the form of a hook, mount for sensors, cable, of a cable itself, of a conductor, of a clip element, clasp, zip fastener, protector or of a sensor. In the exemplary embodiment illustrated with the functional element 20 in the form of a rail or stiffening element, improved guidance of the joint in the applied state is achieved. Arranged on the inner side or rear side 11 of the sheet material is the limiting element 30, which is illustrated by a dashed line. The limiting element 30 is preferably flexible, but not elastic and may be in the form of a film, adhesive layer and elastic textile or the like. There is also the possibility of arranging a plurality of limiting elements 30 one on top of another in order to enhance the limiting action. The limiting element 30 may be materially bonded, for example adhesively bonded or welded, to the sheet material or else integrated inside the sheet material 10, for example encapsulated by casting, encapsulated by foaming or laminated in. The functional element 20 has an outer contour 21 which is inside an outer contour 31 of the limiting element 30, with the result that no elastic sheet material 10 exerts an elastic or an enhanced elastic stretching effect in the region in which the functional element 20 is attached on the outer side or front side 12 of the sheet material 10. In the exemplary embodiment illustrated, the outer contour 31 of the limiting element 30 protrudes beyond the outer contour 21 of the functional element 20 on all sides, in order to ensure that unwanted shear stress does not occur at any point.

FIG. 3 shows an elastic sheet material 10 in the form of a basic material. Connecting devices 50 configured as hook and loop fastener regions, snap fasteners, clasps, loops or the like are arranged or formed on two opposite side edges of the sheet material 10, in order to connect the end regions to one another and to form a closed cross section to, for example, configure the sheet material 10 to afford a bandage 40 with a closed cross section, which bandage can be placed on and taken off again easily and can be adapted in terms of its diameter or circumference. In the sectional illustration on the left, it can be seen that the functional element 20 is applied, for example adhesively bonded, printed or welded, directly to the limiting element 30, wherein the limiting element 30 is fastened on the front side 12 of the sheet material 10. The limiting element 30 is embedded partially in the material of the sheet material 10 and protrudes slightly beyond the front side 12. As an alternative, the limiting element 30 may be integrated completely in the sheet material 10 and finish flush with the front side 12, with the result that a common plane is formed between the front side 12 and the outer side of the limiting element 30, in which plane the functional element 20 is then applied, in particular adhesively bonded or welded. In the illustration on the right in FIG. 3, it can be seen that the outer circumference of the limiting element 30 is larger than that of the functional element 20 and the projection of the functional element 20 is located within the projection of the limiting element 30.

FIG. 4 shows a variant of the sheet material 10. In the sectional illustration, it can be seen that the limiting element 30 is embedded inside the sheet material and the functional element 20 is arranged and fastened on the sheet material 10 on the front side 12. The arrangement of the limiting element 30 inside the sheet material 10 behind the functional element 20 prevents shear forces on the outer side or front side 12 from being transferred when a tensile force is applied over the sheet material 10 and prevents or at least lessens a shear stress in the region of the fastening of the functional element 20. Here, the limiting element 30 likewise has a larger surface area than the functional element 20; the outer contour 21 of the functional element 20 is located inside the outer contour 31 of the limiting element 30.

FIG. 5 shows a further variant, in which, instead of a single limiting element 30, a multiplicity of limiting elements 30, five here, are arranged on the rear side 11, positioned in a strip-shaped manner and connected to the sheet material 10. Such an arrangement is expedient in particular when tensile forces are to be expected only in the longitudinal extent of the limiting elements 30 and of the functional element 20 and/or the limiting element 30 extends predominantly in the longitudinal direction. A grid-like arrangement of strip-shaped limiting elements 30 is likewise possible. Limiting elements 30 are fastened on the rear side 11 of the sheet material 10 in the manner in which they were described in connection with the limiting element 30 from FIG. 3. In the arrangement of the limiting elements 30 that, as illustrated, protrudes slightly inward, said limiting elements may also be formed so as to finish flush with the rear side.

FIG. 6 shows a variant of FIG. 2 with two limiting elements 30, which are arranged at the distal and proximal end regions of the functional element 20 on the rear side 11 or inside the sheet material 10, as indicated by the broken lines. No limiting elements 30 are arranged in the region of the natural axis of the joint, here the axis of the knee joint, in order to permit freedom of movement in this region. There is the possibility that the functional element 30, for example a spring or a rail which is elastic in this region or else an articulated rail, is not connected to the front side 12 or outer side of the sheet material 10 in the region which is not covered by the limiting elements 30, in order thus to allow a relative movement between the functional element 20 and the basic body 10 of the bandage 40 in this region. In this exemplary embodiment, the functional element 20 protrudes in certain regions beyond the outer contour 31 of the limiting element, for example.

A further variant of the invention is shown in FIG. 7, in which the limiting element 30 is arranged on the rear side 11 of the sheet material 10. The limiting element 30 has penetrating elements 32 configured as pins, which penetrate the sheet material 10 and protrude beyond the front side 12. In the region of the penetration, the penetrating elements 32 may have projections, fastening elements, receptacles for clips or similar form-fitting elements, or simply only plate-like or mushroom-like widened portions, in order to achieve a form-fitting fastening by way of the penetrating elements 32 in addition to a materially bonded fastening of the limiting element 30 on the rear side 11. In principle, it is also possible to dispense with a material bond between the limiting element 30 and the sheet material 10. The penetrating elements 32 serve to fasten the functional element 20 or to support the fastening to the sheet material 10. The fastening may be performed in a materially bonded manner on the front side 12 of the sheet material 10 alone or in combination with a form-fitting connection or else material bond with the penetrating elements 32.

FIG. 8 shows a further variant of the invention, in which the limiting element 30 is integrated inside the sheet material 10 and penetrates it completely in the region of the functional element 20. The limiting element 30 may be applied in the form of initially liquid adhesive to a textile basic body in a defined region and penetrate the textile. The functional element 20, e.g. a stiffening element, may be secured to the sheet material 10 by way of the adhesive of the limiting element 30. The adhesive stiffens the textile and prevents movement in the region in which the adhesive hardens. Instead of securing the functional element 20 by way of the adhesive, it is also possible to print said functional element on the limiting element or to apply it by means of an additive production process. The limiting element 30 may finish flush with the sheet material 10 on the front side 12 or project beyond said sheet material, as is shown in FIG. 8. The limiting element 30 preferably finishes flush with the sheet material 10 on the inner side 11.

The limiting element 30 is preferably similarly flexible as the sheet material 10, but has a considerably reduced stretchability.

1. An elastic sheet material with a front side, a rear side situated opposite the front side, and at least one functional element arranged on the front side and connected to the sheet material, wherein a limiting element for limiting the elasticity of the sheet material is arranged on the rear side of the sheet material opposite the functional element and/or on the front side below the functional element or inside the sheet material below the functional element.

2. The sheet material according to claim 1, wherein the limiting element has an outer contour which corresponds to the outer contour of the functional element or encloses the outer contour of the functional element.

3. The sheet material according to claim 1, wherein the limiting element is materially bonded to the sheet material.

4. The sheet material according to claim 1, wherein it is in the form of a textile.

5. The sheet material according to claim 1, wherein the limiting element is in the form of a film, adhesive layer, inelastic textile, penetrating element with projection, conductive textile, band, cable or sensor.

6. The sheet material according to claim 1, wherein a plurality of limiting elements are arranged separate from one another on the sheet material.

7. The sheet material according to claim 1, wherein the limiting element has a flexible form.

8. The sheet material according to claim 1, wherein the functional element is applied, in particular in the form of an elastomer element, to the sheet material by an additive production process.

9. The sheet material according to claim 1, wherein the functional element is in the form of a spring, rail, stiffening element, component receptacle, duct, hook, sensor mount, cable guide, cable, conductor, clip element, clasp, zip fastener, protector, sensor, cushion and/or air cushion.

10. A bandage made of a sheet material of claim 1.

11. The bandage according to claim 10, wherein the bandage has a sheath-like form with a closed cross section or is formed from a sheet-like basic body with connecting devices arranged at opposite end regions.

12. A method for producing a sheet material according to claim 1, wherein the functional element is connected to the sheet material and at least one limiting element is introduced between the functional element and the sheet material and/or on that side of the sheet material which is situated opposite the functional element, and is connected to the sheet material, and in that the limiting element at least lessens the elasticity of the sheet material.

13. The method according to claim 12, wherein the functional element is applied to the sheet material or the limiting element in an additive production process.

14. The method according to claim 12, wherein the limiting element is connected to the sheet material over its entire surface area.

15. The method according to claim 1, wherein the functional element is arranged inside the outer contour of the limiting element.

16. The method according to claim 1, wherein the limiting element is materially bonded to the sheet material.

17. An elastic sheet material, the sheet material having:
a front side;
a rear side situated opposite the front side; and
at least one functional element arranged on the front side and connected to the sheet material;
wherein a limiting element for limiting the elasticity of the sheet material is materially bonded to the rear side of the sheet material opposite the functional element and/or on the front side below the functional element or inside the sheet material below the functional element, the limiting element having an outer contour which corresponds to an outer contour of the at least one functional element.

18. The sheet material according to claim 17, wherein the limiting element has a flexible form.

19. The sheet material according to claim 1, wherein the functional element is applied, in particular in the form of an elastomer element, to the sheet material by an additive production process.

20. An elastic sheet material in the form of a textile, the sheet material having:
a front side;
a rear side situated opposite the front side; and
at least one functional element arranged on the front side and connected to the sheet material;
wherein a plurality of limiting elements for limiting the elasticity of the sheet material are materially bonded to the rear side of the sheet material arranged separate from one another on the sheet material opposite the functional element and/or on the front side below the functional element or inside the sheet material below the functional element, the limiting element having an outer contour which corresponds to an outer contour of the at least one functional element.
